(19) United States
(12) Patent Application Publication
(10) Pub. No.: US 2024/0050619 A1
(43) Pub. Date: Feb. 15, 2024

(54) ULTRAVIOLET AIR DISINFECTION SYSTEM

(71) Applicant: Peter KOCHAN, Bosaca (SK)

(72) Inventor: Peter KOCHAN, Bosaca (SK)

(21) Appl. No.: 18/271,063

(22) PCT Filed: Jan. 6, 2021
(86) PCT No.: PCT/SK2021/050001

§ 371 Date: Jul. 6, 2023

(51) Int. Cl. A61L 9/20; B08B 7/00; A61M 16/10

(52) U.S. Cl. CPC A61L 9/20; B08B 7/0057; A61M 16/10; A61L 2209/12; A61L 2209/11; A61M 2209/088; A61M 2205/8243; A61M 2205/3592; A61M 2205/50

(57) ABSTRACT

Wearable UV air disinfection system in a compact and fully portable form factor. Practical air disinfection system may be worn by a user to kill or deactivate germs, viruses or other pathogens, which are located in the air to be breathed by the user. Air being inhaled or exhaled by the user is exposed to Ultra-Violet C-band (UVC) radiation. This UVC radiation is lethal to undesirable germs, viruses and other pathogens. In this manner, pathogen-free air is being inhaled or exhaled by the user.

### TECHNICAL FIELD

The invention generally relates to using ultraviolet radiation to disinfect air.

### BACKGROUND ART

Ultraviolet (UV) germicidal irradiation for the purpose of disinfection has been an accepted practice since mid-20th century in medical field, or for drinking and wastewater sterilization. High-intensity shortwave ultraviolet light is commonly used for disinfecting smooth surfaces such as dental tools. Unfortunately, existing UV-light sources for biocidal applications are typically fluorescent UV light bulbs, deuterium or xenon-arc excimer lamps. These lamps and bulbs are large and usually require the presence of a ballast and AC power source for stable operation. See, for example, U.S. Pat. No. 5,817,276 assigned to Steril-Aire. Size and power requirements of such high-intensity shortwave ultraviolet disinfectant systems make them unsuitable for portable use and are not known in this context.

Conventional germicidal UV light applications usually use UV light sources in 250 nm-280 nm wavelength, allowing them to disinfect unoccupied spaces or enclosed areas, however using them in close proximity to humans is not possible since it is well known that exposure to these wavelengths is a health hazard, causing eye diseases, skin cancer and other health problems.

Attempted portable solutions in this field include a protective face mask combining mechanical filtering element with 265 nm UV-C LEDs, however due to the health hazard associated with the 265 nm UV wavelength, the UV light source must be physically enclosed in a chamber, restricting the airflow, and only disinfecting the air passing through this chamber, whereas if the mask is not properly face-fitted, a significant amount of air can leak through the periphery of the mask allowing inhalation of airborne pathogens by the person wearing it, significantly reducing the mask's effectiveness and increasing the risk of respiratory infection. Many facemasks and respirators cannot be re-used repeatedly, and their disposal is problematic and poses potential environmental danger and burden. Furthermore, facemasks and respirators are generally uncomfortable to wear, which may discourage people from using them, they usually muffle the voice and can make speech less intelligible, the mechanical or other filtering elements in them usually only capture some of the pathogens and need frequent cleaning or replacement, and some people are unable to wear facemasks and respirators due to breathing difficulties, allergies, or other reasons.

From the discussion above it should be apparent that there is a need for air disinfection against airborne viral or bacterial infection that provides protection for persons on the move and in a variety of environments, without them having to wear a facemask or other protective device that could pose an obstruction to breathing or speech intelligibility. The present invention satisfies this need.

### SUMMARY OF INVENTION

Portable air disinfection system that is worn by a user and irradiates the airflow with energy in the electromagnetic spectrum, while the air is being inhaled by the user. The irradiated energy is sufficient to substantially cleanse the inhaled air of most airborne viruses and bacteria.

Recent third-party research has shown that the far-UVC range of 200 nm-230 nm has similar anti-microbial and anti-viral properties as conventional germicidal UV lights, but without producing the corresponding negative health effects. Reason for this is that light in this far-UVC wavelength range has a very limited biological penetration depth. Although the penetration is limited, it is still much larger than the size of viruses and bacteria, therefore far-UVC light is able to kill these pathogens just like conventional germicidal. UV light.

Since shortwave far-UVC photons at 200-230 nm range have germicidal effect without penetrating or damaging live human cells, making far-UVC sanitizing light safe for humans, and while potentially harmful ozone is only produced at wavelengths below 200 nm, the range of 200-230 nm can be safely used in air disinfection and personal protection devices.

Presented invention applies this knowledge in an air disinfection system that uses light in the 200-230 nm far-UVC range to irradiate and disinfect the air being inhaled or exhaled by the user, and presented embodiments represent some of the potential industrial applications in the form of portable air disinfection devices that reduce risk from airborne pathogens.

Accordingly, it is an object of the present invention to provide a UVC air disinfection system for killing germs, viruses, and other pathogens by the use of radiation in a compact and portable form factor.

It is still another object of the present invention to minimize the obstructions to airflow of inhaled or exhaled air of the user of the portable air disinfection system.

It is still another object of the present invention to minimize exposure to airborne pathogens for anyone near an already infected user by disinfecting the air being exhaled by the user of the portable air disinfection system.

It is still another object of the present invention to allow total freedom of movement of the user of the portable air disinfection system.

These and other features and advantages of the present invention further enhancing and advancing the technology in the art will become apparent upon further review of the following specification, drawings, and claims. It should be understood, however, that the detailed description and the specific examples while indicating preferred embodiments of the invention are given by way of illustration only, since various changes and modifications within the spirit and scope of the invention will become apparent to those skilled in the art from this detailed description.

Embodiments of the invention pertain to a portable system that is worn by a user and irradiates the airflow with energy in the UVC electromagnetic spectrum while the air is being inhaled by the user. The irradiated energy is sufficient to substantially cleanse the air of airborne viruses and the like prior to inhalation. The system includes a portable radiation source with a power supply to generate the energy for irradiation, and an electronic control module managing the operation of the system. The air is irradiated upon entering the airways of the upper respiratory system (nose, mouth) such that airborne germs in the airflow are substantially destroyed or rendered harmless before the air enters into pharynx and lower respiratory system and germs in it potentially cause an infection. Viral and bacterial exposure is defeated at the point of inhalation, thus reducing the threat of infection independent of the airborne bacteria type, virus strain or mutation of the virus.

Electromagnetic radiation for the air disinfection is generated at the UVC light source and is directed from the light source to the area in front of user's nose and mouth preferably by an optical diffuser, optionally including a conduit such as fiber optics.

Source for the UVC light for air disinfection in the embodiments is a laser module providing light emission with a wavelength between 200 and 230 nm, in a portable form factor that can be carried by the user. Optionally, this laser module may comprise of a laser diode emitting wavelength between 400 and 460 nm, a frequency doubling element such as BBO crystal, providing light emission with a wavelength between 200 and 230 nm, and a compact wavelength filter ensuring pure ultraviolet emission. Alternatively, laser diode of other wavelength may be used as long as emitted radiation wavelength between 200 and 230 nm is achieved, for example by a third-harmonic generation or sum-frequency mixing.

Apparatus can be powered by a standard or a rechargeable battery, optionally by any other suitable power source so as to preferably provide a power capacity of at least 1 microwatt-hour. If a rechargeable battery is used, it can be recharged through a charging port or wirelessly using electromagnetic induction. Alternatively, the apparatus may also be powered by an external power supply, such as USB power bank, or by wireless power transfer.

Function of the system is managed by an electronic control module, which may comprise a computer, optionally also an antenna to communicate with external devices, allowing the user to control and manage the air disinfection system both directly and indirectly by an externally connected device such as a smartphone. This also allows a function of automatically turning on the air disinfection system when leaving biosafe environment such as personal home or car, or when entering potentially hazardous environment. System functionality or power source status can be confirmed by either integrated visual or acoustic indicators, or remotely by means of externally connected device such as a smartphone.

In some embodiments, one or more of the apparatuses, systems or devices described herein can be used, and their use in combination with other forms of disinfection or mechanical filters are within the scope of embodiments of the invention.

### Technical Problem

Personal protection against airborne viral or bacterial infection currently requires wearing a facemask or a respirator, and existing ultraviolet disinfection devices are usually large, cannot be worn on body of the user, and use ultraviolet light wavelengths that are harmful to humans.

### Solution to Problem

Miniaturized battery-powered high-intensity 200-230 nm shortwave UV air disinfection system in a compact easy-to-wear and fully portable form factor that may be worn by a user to kill or deactivate germs, viruses or other pathogens, which are present in the air to be breathed by the user.

### Advantageous Effects of Invention

Invention provides protection for persons in a variety of environments as they move about.

Invention provides personal protection against airborne pathogens without having to wear a facemask posing an obstruction to breathing.

Invention provides personal protection against airborne pathogens without having to wear a facemask that makes speech less intelligible.

Invention provides protection for persons in a vicinity of an infected person exhaling air containing pathogens.

### BRIEF DESCRIPTION OF DRAWINGS

Various other objects, features and attendant advantages of the present invention will become fully appreciated as the same becomes better understood when considered in conjunction with the accompanying drawings, in which like reference characters designate the same or similar parts throughout the several views.

For a better understanding of the embodiments described, herein and to show more clearly how they may be carried into effect, reference will now be made, by way of example only, to the accompanying drawings which show at least one exemplary embodiment, and in which:

FIG. 1 is a perspective illustration of an embodiment where the apparatus is fully integrated in the glasses worn by the user.

FIG. 2 is a perspective illustration of an embodiment where the apparatus is attached to the glasses worn by the user,

FIG. 3 is a perspective illustration of an embodiment where the apparatus is integrated in an ear-worn assembly.

### DESCRIPTION OF EMBODIMENTS

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular.

In the following description, specific details are set out to provide examples of the claimed, subject matter. However, the embodiments described below are not intended to define or limit the claimed subject matter.

It will be appreciated that, for simplicity and clarity of illustration, where considered appropriate, reference numerals may be repeated among the figures to indicate corresponding or analogous elements or steps. Numerous specific details are set forth in order to provide a thorough understanding of the exemplary embodiments of the subject matter described herein. However, it will be understood by those of ordinary skill in the art that the embodiments described herein may be practiced without these specific details. In other instances, well-known methods, procedures and components have not been described in detail so as not to obscure the present subject matter. Furthermore, this description is not to be considered as limiting the scope of the subject matter in any way but rather as illustrating the various embodiments.

Indeed, it should be understood that various changes may be made in the form, details, arrangement and proportions of the parts. Such changes do not depart from the scope of the invention which comprises the matter shown and described herein and set forth in the appended claims.

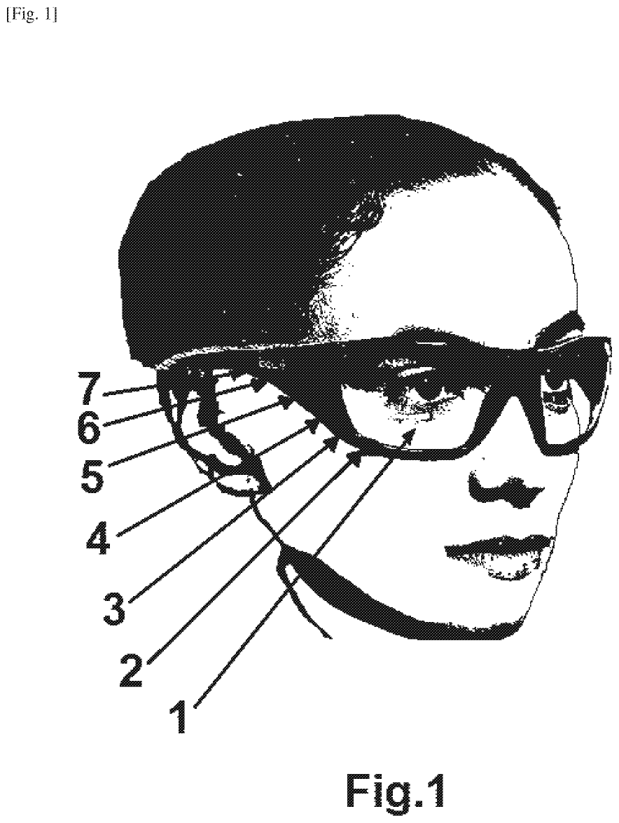

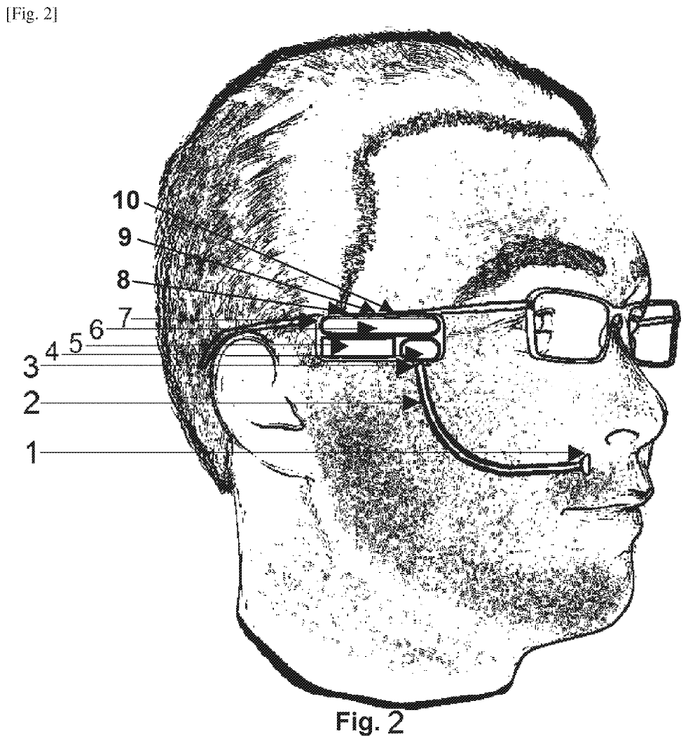

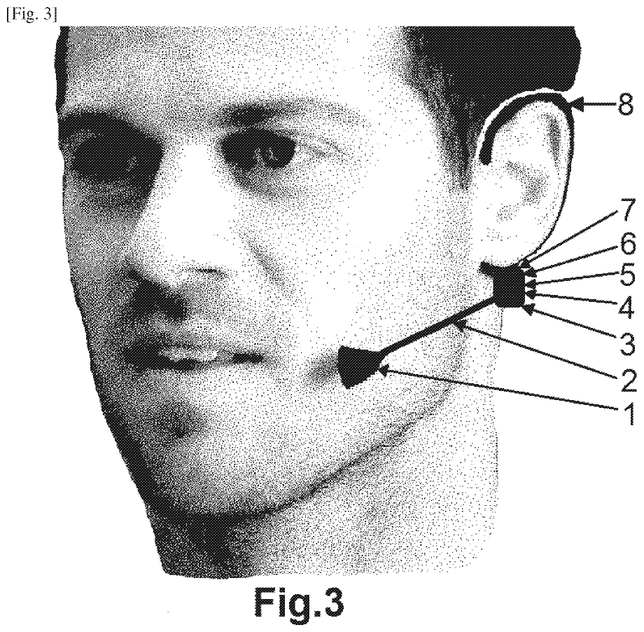

As shown on FIG. 1, FIG. 2, and FIG. 3, the air disinfection system comprises of a wearable housing of the apparatus, a battery or other power supply, USB-C port for connecting external power supply or battery charging, electronic control module, irradiation source, light diffusing element, optionally also a flexible fiber optic conduit.

In an embodiment shown on FIG. 1, the main body of the apparatus 7 is integrated inside the plastic frame of user's glasses, containing a battery 6, for example an 18650 type rechargeable lithium-ion battery, electronic control module 5 comprising a computer and an internal antenna for communication with external device such as smartphone, with an external power switch, a USB-C port 4, and a LED light serving as visual function indicator, irradiation source 3 is a frequency-doubled ˜445 nm laser module outputting light through second harmonic generation in the 222 nm UVC wavelength, coupled with an optical diffuser 2, directing the UVC light toward user's nose and mouth. Optionally, the lenses in the glasses may contain an UV film 1 for additional protection.

In an embodiment shown on FIG. 2, the main body of the apparatus 10 may be an oval shaped plastic casing about 70 mm in length, 20 mm in width, and 35 mm in depth, attached to the side frame of user's glasses by a plastic clip 9, and containing a battery 6, for example an 18650 type lithium-ion battery, electronic control module 5 comprising a computer and an internal antenna, with an external power switch 8, a USB-C port 7, and a LED light serving as visual function indicator, irradiation source 4 is a frequency-doubled ˜445 nm laser module outputting light through second harmonic generation in the 222 nm UVC wavelength, coupler 3 connects the main apparatus body with the proximal end of the flexible conduit 2, wherein this conduit is passing alongside user's cheek toward the nose, directing the UVC light via fiber optic toward the directional diffuser 1 at the distal end located near user's nose and mouth. Optionally, a side-emitting optical fiber can be used instead of a diffuser, such as for example a Corning® or Fibrance® light diffusing fiber.

In an embodiment shown on FIG. 3, the main body of the apparatus 7 may be an oval shaped casing about 40 mm in length, 20 mm in width, and 20 mm in depth, attached to user's ear with a plastic clip 8, containing a battery 6, for example a CR123 type rechargeable lithium battery, electronic control module 5 with an external power switch, comprising a computer and an internal antenna, a USB-C port 4, and a LED light 3 serving as visual function indicator, flexible conduit 2 containing power supply cabling, holding the 222 nm wavelength laser module 1 coupled together with a diffuser near user's nose and mouth.

It will of course be appreciated by those skilled in the art that many variations of the described embodiments would be possible within the scope of the invention defined by the claims herein.

### EXAMPLES

Commercially available miniaturized 5 Watt ˜445 nm laser, attached to user's glasses frame, powered by 18650 battery and coupled with frequency-doubling BBO crystal producing through second-harmonic generation UVC light in the 222 nm wavelength, optically coupled by a multimode fiber coupler with a biconvex lens reducing the output beam diameter into a 400 μm/440 μm/480 μm (core/cladding/coating) solarization resistant fiber, on the distal end with a shaped glass diffuser made by Schott, produces sufficient far-UVC irradiance to achieve significant inactivation (over 90%) of most airborne viruses at the exposure rate required for the volume and speed of air entering upper airways.

Dose required to kill viruses and bacteria in the air with far-UVC is slightly higher than with conventional UVC at 254 nm or higher wavelengths; however, the lack of harmful biological effects on user's body with far-UVC allows the possibility to safely use it in close proximity to human skin.

Recent advances in far-UVC laser technology have shown promise for further shrinking both the size and the cost of the laser modules to be better suited for widespread adoption, new solid state light sources such as aluminum gallium nitride laser diodes emitting far UVC wavelengths will eliminate the need for using frequency-doubling crystals and will allow simplification of the used laser module, and latest developments in the light diffusing technology, as well as developments in minimizing solarization effects reducing UVC transmittance in optical fibers, will further enhance system efficiency, allowing smaller system size, and requiring lower power consumption as well as longer runtime per battery charge.

### INDUSTRIAL APPLICABILITY

Invention can be easily manufactured and can be used in any kind of industry by anyone needing protection from airborne pathogens.

### REFERENCE SIGNS LIST

Reference to Deposited Biological Material

Sequence Listing Free Text

### CITATION LIST

### Patent Literature

PTL 1:

### Non Patent Literature

NPL 1:

1. An air disinfection apparatus carried on an individual person, comprising of:
at least one laser light source outputting light in the 200-230 nm UVC light spectrum, wherein the electromagnetic radiation is directed toward the air being inhaled by the person, treating the air with a radiated energy of the electromagnetic spectrum such that at least one airborne virus in the air being inhaled by the person is substantially rendered in-effective,
an electronic control module,
a power supply,
a wearable housing.

2. The air disinfection apparatus as defined in claim 1, wherein the apparatus is powered by a non-rechargeable battery inside the housing.

3. The air disinfection apparatus as defined in claim 1, wherein the apparatus is powered by a rechargeable power source inside the housing.

4. The air disinfection apparatus as defined in claim 1, wherein the apparatus is powered by an external power supply.

5. The air disinfection apparatus as defined in claim 1, wherein the apparatus is powered by a wireless power transfer.

6. The air disinfection apparatus as defined in claim 1, wherein the light source is coupled to at least one optical fiber extending therefrom.

7. The air disinfection apparatus as defined in claim 1, wherein the apparatus comprises at least one diffuser for directing the radiation toward the air being inhaled by the user.

8. The air disinfection apparatus as defined in claim 1, wherein the light source comprises a fiber optic conduit that includes an area of reduced transmissivity and an exposed surface from which the electromagnetic radiation is emitted.

9. The air disinfection apparatus as defined in claim 1, wherein the electronic control module comprises an antenna for wireless connection to an external device for transmitting and receiving information.

10. The air disinfection apparatus as defined in claim 1, wherein the electronic control module comprises an integrated computer managing the system functionality based on information received from an external device.
